(19) United States
(12) Patent Application Publication
(10) Pub. No.: US 2021/0187666 A1
(43) Pub. Date: Jun. 24, 2021

(54) METHOD FOR PRODUCING AN IMPLANT, AND IMPLANT PRODUCED BY SAID METHOD

(71) Applicant: BIOTRONIK AG, Bülach (CH)

(72) Inventors: Stefan Remund, Bern (CH); Thorsten Kramer, Solothurn (CH); Beat Neuenschwander, Burgdorf (CH); Beat Jaeggi, Biel (CH)

(21) Appl. No.: 17/268,827

(22) PCT Filed: Aug. 20, 2019
(86) PCT No.: PCT/EP2019/072236

§ 371 Date: Feb. 16, 2021

(30) Foreign Application Priority Data
Aug. 24, 2018 (EP) 18190805

(51) Int. Cl. B23K 26/38; A61F 2/91; A61L 31/02

(52) U.S. Cl. CPC B23K 26/38; A61F 2240/001; A61L 31/022; A61F 2/91

(57) ABSTRACT

A method for producing an implant, such as a stent, includes providing an implant blank, such as a stent preform, formed from a degradable material, preferably a magnesium alloy. Recess are introduced into the implant blank through laser beam ablation using laser radiation in the form of pulse bundles, each pulse bundle having at least two pulses.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2019/072236, which was filed Aug. 20, 2019, which application claimed priority from European Application EP 18190805.4, which was filed Aug. 24, 2018.

### FIELD OF THE INVENTION

A field of the invention is medical implants, in particular stents, and methods for production of such implants.

### BACKGROUND

In stent manufacturing processes, laser radiation is often used to cut the stent. In particular, pulsed laser radiation is typically used for material removal/cutting.

The laser pulses are specified by their pulse duration and their pulse energy. The pulse duration is typically specified as the Full Width at Half Maximum (FWHM), and the width of the pulse is measured at half pulse height. For ultra-short pulses with pulse durations in the range of from a few femtoseconds to a few picoseconds, the pulse shape can be considered to be time-symmetrical The energy of a pulse is calculated formally by the integration of the temporal course of the pulse power (the pulse shape) over time and is specified in joules (J), millijoules (mJ) or microjoules (pJ). It can also be calculated as an averaged value from the quotient of the mean power and the repetition rate.

The repetition rate or also pulse sequence frequency defines how many individual pulses per unit of time are emitted by the laser. It is specified in hertz (Hz), kilohertz (kHz) or megahertz (MHz).

Instead of an individual pulse, it is possible in the case of some laser beam sources, at a given repetition rate, to divide the pulse energy of an individual pulse into a sequence of individual pulses, or what are known as pulse bundles or pulse bursts.

A pulse bundle or pulse burst (PB) is a sequence of individual pulses which consists of at least two successive individual pulses (P), wherein the time interval (B) of the individual pulses of a pulse bundle is shorter than the time interval (C) of two pulse bundles, i.e. the reciprocal value of the repetition rate of the laser. (FIG. 1). In the case of a pulse bundle the time interval between each two adjacent pulses of a pulse bundle is shorter than the time interval between the last pulse of a pulse bundle and the first pulse of the next pulse bundle. Within the scope of this application the terms pulse bundle and pulse burst are used synonymously.

Depending on the structure of the laser, in the case of ultra-short-pulsed laser beam sources a distinction is made between Master-Oscillator-Power-Amplifier (MOPA) systems and Regenerative Amplifier (Regen) systems. The pulse bundle for MOPA systems is generated in what is known as the pulse picker, which not only allows an individual pulse to pass, but also a plurality of pulses (typically 2 to 10) with the selected repetition rate. The time interval between the pulses in the pulse bundle therefore corresponds to the inverse of the frequency of the laser oscillator (Master Oscillator) and lies typically in the range of from 20 MHz to 100 MHz. Such laser sources nowadays already reach frequencies in the GHz range. The oscillator frequency is therefore much higher than typical repetition rates of less than 1 MHz, which are currently used for processing. Expressed more simply, the laser source produces pulsed laser radiation with a high frequency. The pulse picker selects, from a continuous sequence of pulses, the individual pulse bundles with the desired repetition rate of the laser and the individual pulses of a pulse bundle. The pulse picker thus defines the repetition rate of the laser (time interval of the pulse bundles) and the number of pulses of a pulse bundle.

The individual pulses or pulse bundles are then amplified in the amplifier (Power Amplifier). In modern ultra-short-pulse laser systems it is possible to adjust the amplification factor individually for each pulse of the pulse bundle. There is also the possibility to set the amplification of a pulse to zero and to thus blend out the pulse. The individual pulse number and the time interval between the individual pulses of a pulse bundle are thus controlled. In principle, each pulse bundle can thus be formed individually.

Ultra-short-pulse laser systems are generally used for material removal (ablation). The ablation rate in this case denotes the ablated volume per unit of time and is specified typically in cubic millimetres per minute (mm³/min).

The ablation efficiency or the specific ablation rate is understood to mean the ablation rate based on the introduced mean laser power, that is to say the ablated volume per energy unit. It is therefore specified in mm³/(min W) or also in μm³/(μJ).

During the processing with laser radiation with individual pulses at a selected repetition rate, the maximum ablation efficiency is delimited, amongst other things, by the creation of zones and craters on the workpiece surface, which negatively influence the absorption of the laser radiation. The ablation process, in the worst-case scenario, can come to a complete halt.

The use of individual pulses at a moderate medium power and at not-too-high repetition rates generally leads to the best-possible surface quality in almost all examined materials.

Generally, it is desirable in the case of laser beam ablation processes of this kind to increase the ablation rate while maintaining a constant surface quality. In addition, it is expedient and desirable at the same time not to reduce, or even increase, the ablation efficiency.

An increased ablation rate, alongside a constant quality, can be achieved, however, only if the repetition rate of the laser and thus its mean power are increased accordingly. Such an increase is delimited in particular by two factors. On the one hand the mean power in the case of commercial ultra-short-pulsed laser beam sources, which are usually used, is limited to approximately 100 W, and on the other hand a number of tests have shown that, when the repetition rate is increased, the subsequent pulses are influenced increasingly for example by what is known as shielding, i.e. the interaction or partial shielding of the laser radiation of a pulse with the already ablated material of the previous pulse, and therefore the ablation efficiency decreases.

### SUMMARY OF THE INVENTION

A preferred method for producing an implant, such as a stent, is disclosed, includes providing an implant blank, such as a stent preform, formed from a degradable material, preferably a magnesium alloy. Recess are introduced into the implant blank through laser beam ablation using laser radiation in the form of pulse bundles, each pulse bundle having at least two pulses.

### BRIEF DESCRIPTION OF THE DRAWINGS

Further features and advantages of the invention will be explained in the description of the figures depicting exemplary embodiments of the invention, provided with reference to the figures, in which:

FIG. 1 shows a schematic depiction of pulse bundles (pulse bursts) with three pulses; and

FIG. 2 shows the ablation efficiency via the energy irradiated into the material per area ϕ₀ in the case of a magnesium alloy according to the invention and in the case of pulse bundles having three, four and five pulses as compared to an individual pulse.

### DETAILED DESCRIPTION OF THE PREFERRED EMBODIMENTS

The invention provides a method for producing an implant, in particular a stent, from a degradable material, in particular a magnesium alloy, which has an increased ablation efficiency. Preferred embodiments concern methods for producing an implant, in particular a stent, from a degradable material, in particular from a magnesium alloy, and to an implant, in particular stent, produced by the method from such a material, in particular such an alloy. The present invention is described primarily on the basis of the example of a method for producing a stent from a magnesium alloy. However, the present invention is not limited to this application, but is suitable for producing an implant from any degradable material. A degradable material within the scope of this application shall be understood to mean a material which is broken down (degraded) in the body. Implants formed from degradable material (biodegradable implants) maintain their structural integrity over a period of time lasting from hours/days, through months, to several years, and are then broken down (resorbed) by the body. Degradable materials of this kind are known to a person skilled in the art, for example some metal alloys, such as magnesium, iron or zinc alloys, or degradable polymers, such as polylactides.

Within the frame of this application laser beam ablation means the use of a laser beam to form a recess in a workpiece. Thereby the thickness of the workpiece is reduced by the recess. This is in contrast to laser beam cutting where the laser beam is used to cut through a workpiece.

Typically, a stent includes a general cylindrical body including various meandering struts. If laser beam cutting is used to produce such a stent from a cylindrical workpiece, several cut-outs through-openings are made in the cylindrical workpiece. The remaining material forms is the struts of the stent. The laser beam cuts through the cylindrical workpiece. If laser beam ablation is used to produce such a stent, a series of recesses are created in the cylindrical workpiece. The laser beam ablation provides a first recess at a first position. Thereby the thickness of the workpiece is reduced at the first position. At a later point in time a second recess is provided at the same first position, which further reduces the thickness at the first position. This procedure is repeated layer by layer until there is no further material at the first position. Thereby a cut-out through-opening is produced. The remaining material of the cylindrical workpiece forms the struts.

We have surprisingly found, with regard to the ablation of magnesium alloys, that in particular it is possible to respond to the interaction of successive individual pulses signifying a reduction of the ablation efficiency or a reduction of the processing quality by the use of pulse bundles (pulse bursts). The individual pulses within a pulse bundle can be set individually in respect of their magnitude, i.e. in respect of the energy of each individual pulse, and time interval (in multiples of the inverse of the frequency of the laser oscillator) such that not only is the negative influence compensated, but the ablation efficiency can be increased. Since, in addition, the repetition rate does not have to be increased to the same extent as in the case of individual pulses, the interactions between the individual pulse bundles can be reduced or even completely avoided.

Due to the adapted use of pulse bundles during the ablation with ultra-short-pulsed laser radiation, in particular of metals, the specific ablation rate, also referred to as ablation efficiency, i.e. the volume ablated per unit of time and introduced mean laser power, can be increased.

In the case of ablation with ultra-short-pulsed laser radiation, various effects occur at the workpiece surface and over the surface and influence the ablation process. These can be in particular modifications of the surface or shieldings by ablated particles. By selective use of pulse bursts in a configuration that counteracts the mechanisms which negatively influence the ablation process, the ablation efficiency in the case of magnesium alloys can be increased, what's more even beyond the value of an individual pulse.

A comparable effect is produced with other degradable materials, in particular with degradable polymers, such as polylactides, or other metal alloys, such as iron or zinc alloys.

In accordance with a preferred embodiment it is provided that each pulse bundle of the laser radiation used for the ablation has three pulses, in particular precisely or merely three pulses. In this regard it has been demonstrated that, in the case of a magnesium alloy, compared to an individual pulse the use of a pulse bundle with three pulses increases the ablation efficiency from 0.19 mm³/(min·W) by 68% to 0.32 mm³/(min·W).

In accordance with a further preferred embodiment it is provided that each pulse bundle of the laser radiation used for ablation has four pulses, in particular precisely or merely four pulses. In this regard it has been demonstrated that, in the case of a magnesium alloy, compared to an individual pulse the use of a pulse bundle with four pulses increases the ablation efficiency from 0.19 mm³/(min·W) by 142% to 0.46 mm³/(min·W).

In accordance with a further preferred embodiment it is provided that each pulse bundle of the laser radiation used for ablation has five pulses, in particular precisely or merely five pulses. In this regard it has been demonstrated that, in the case of a magnesium alloy, compared to an individual pulse the use of a pulse bundle with five pulses increases the ablation efficiency from 0.19 mm³/(min·W) by 195% to 0.56 mm³/(min·W).

Furthermore, it is provided in accordance with an embodiment of the invention that the time interval between each two adjacent pulses of a pulse bundle lies in a range of from 10 ps to 100 ns, preferably 10 ns to 50 ns (100 MHz to 20 MHz), and in particular is 12 ns.

The use of pulse bundles in which adjacent pulses of the pulse bundle have a time interval of approximately 12 ns leads advantageously to a local melting, of limited depth, of the surface of the material of the implant blank, in particular the stent preform, and therefore already the first unevennesses and inhomogeneities are compensated and the formation of cones and craters is prevented.

It is furthermore provided in accordance with an embodiment of the invention that the pulse duration of the pulses of a pulse bundle lies in the range of from 0.200 ps to 20 ps and is in particular 10 ps. Pulse durations for the pulses of a pulse bundle likewise advantageously lie in the range of from 0.2-0.5 ps, 0.8-0.9 ps, 6-12 ps or around 20 ps.

It is furthermore provided in accordance with an embodiment of the invention that the repetition rate of the pulse bundles lies in a range of from 0.1 MHz to 20 MHz and in particular is 500 kHz. Repetition rates for the pulse bundles are likewise advantageously in the region of 0.1-1 MHz, 2 MHz or 8 MHz.

It is furthermore provided in accordance with an embodiment of the invention that the wavelength of the laser radiation lies in the infrared range of from 1000 nm to 1500 nm, in particular is 1026-1090 nm. In some embodiments, however, it may also be advantageous to use a laser source with a laser radiation in the range of 513-545 nm or 342-363 nm.

Furthermore, the implant blank, in particular stent preform, in accordance with an embodiment can be tubular and extends along a longitudinal axis or cylinder axis.

In accordance with an embodiment of the invention the implant blank, in particular stent preform, as it is acted on with the laser radiation in order to form the recesses, is rotated and/or is moved in the direction of the longitudinal axis.

In accordance with an embodiment of the invention it is also possible (in particular as an alternative to a movement of the implant blank, in particular stent preform, along its longitudinal axis) that the laser radiation in order to form the recesses is moved along the longitudinal axis (for example a head of a cutting tool emitting the laser light can be moved along the longitudinal axis of the implant blank, in particular stent preform).

In accordance with a further aspect of the present invention an implant, in particular a stent, is disclosed which has been produced by means of the method according to the invention.

The present invention is particularly suitable for cutting and structuring vascular supports/stents. Since the processing is performed layer-by-layer by material ablation, in principle any geometry advantageously can be processed.

With regard to the processing of vascular supports/stents, the increase in efficiency according to the invention results in a decisive reduction of the processing duration, with a simultaneous increase in the flexibility as compared to the conventional thermal laser beam cutting.

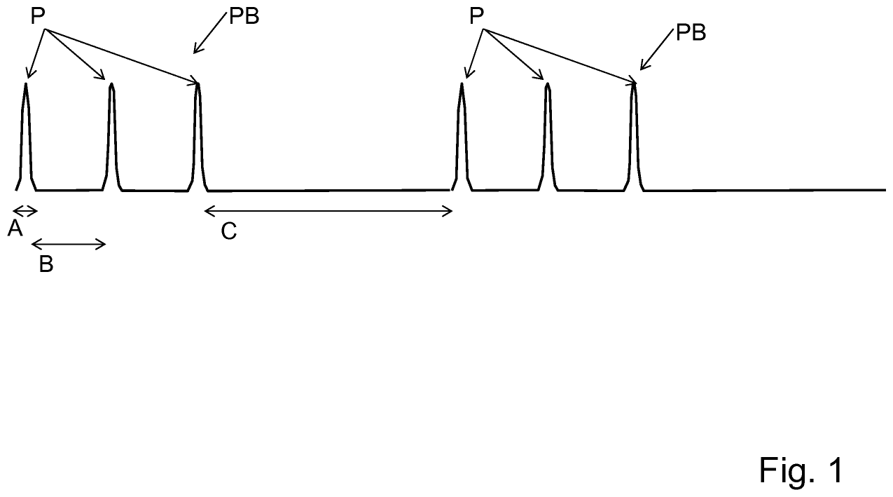

FIG. 1 shows a schematic depiction of pulse bundles (PB) with three pulses P as can be used to cut a stent preform formed from a magnesium alloy of the type described herein. The pulse bundle operation of the laser used for ablation/cutting is characterised in that the time intervals B of two adjacent pulses P of a pulse bundle PB are shorter than the time interval C between the last pulse P of a pulse bundle PB and the first pulse P of the subsequent pulse bundle PB.

In accordance with the invention a laser beam with pulse bundles PB of this kind is directed to the stent preform and a plurality of recesses are cut out or ablated from the stent preform, which in particular is tubular, so as to obtain in particular a stent having a plurality of cells, which are delimited by struts of the stent.

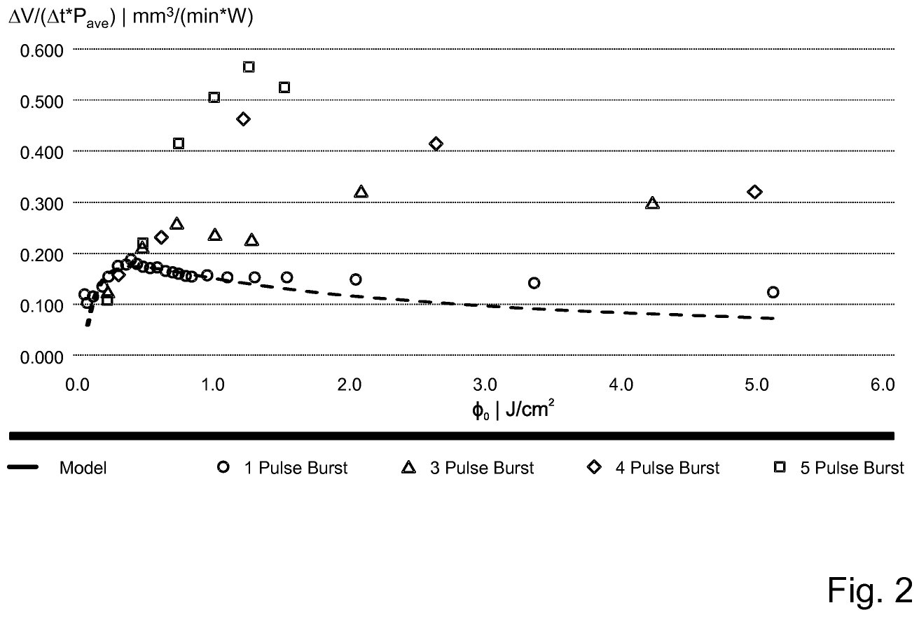

As is shown in FIG. 2, the ablation efficiency, which is plotted on the Y-axis or ordinate (the energy irradiated into the stent material per area #4 is plotted on the abscissa), can be increased when laser radiation with pulse bundles having three, four or five pulses instead of individual pulses is used to ablate the stent material (here for example a magnesium alloy).

The pulse duration A used here for the pulses P of the pulse bundle PB is A=10 ps. The interval B of the pulses B=12 ns, and the interval C of the pulse bundles PB is C=2 ps. This corresponds to a repetition rate (pulse sequence frequency) of the pulse bundle PB of 500 kHz. Laser radiation of wavelength λ=1064 nm is used (see FIG. 1).

Commercial laser systems such as LUMENTUM, Amplitude Systemes or Coherent can be is used order to produce the pulse bundles PB according to the invention.

1. A method for producing an implant, in particular a stent, comprising the steps:
providing an implant blank formed from a degradable material, and
introducing recesses into the implant blank through laser beam ablation
using laser radiation in the form of pulse bundles (PB), wherein each pulse bundle (PB) comprises at least two pulses (P).

2. The method according to claim 1, wherein each pulse bundle (PB) is formed by three pulses (P).

3. The method according to claim 1, wherein each pulse bundle (PB) is formed by four pulses (P).

4. The method according to claim 1, wherein each pulse bundle (PB) is formed by five pulses (P).

5. The method according to claim 1, wherein a time interval (B) between each two adjacent pulses (P) of a pulse bundle (PB) lies in a range of from 10 ps to 100 ns.

6. The method according to claim 1, wherein a pulse duration (A) of each pulse (P) of a pulse bundle (PB) lies in the range of from 0.200 ps to 20 ps.

7. The method according to claim 1, wherein a repetition rate of the pulse bundles (PB) lies in the range of from 0.1 MHz to 20 MHz.

8. The method according to claim 1, wherein a wavelength of the laser radiation lies in the infrared range of from 1000 nm to 1500 nm.

9. The method according to claim 1, wherein the implant blank is tubular and extends along a longitudinal axis.

10. The method according to claim 9, comprising rotating and/or moving the implant blank in the direction of the longitudinal axis while introducing the recesses through laser beam ablation.

11. The method according to claim 10, wherein the implant blank is moved along the longitudinal axis while introducing the recesses through laser beam ablation.

12. (canceled)

13. The method according to claim 5, wherein the time interval (B) lies in a range of between 10 ns and 50 ns.

14. The method according to claim 6, wherein the pulse duration (A) is 10 ps.

15. The method according to claim 7, wherein the repetition rate lies in the range of 500 kHz to 5 MHz.

16. The method according to claim 8, wherein the wavelength of the laser radiation lies in the range of 1026-1090 nm.

17. The method according to claim 1, wherein the implant blank comprises a stent preform.

18. The method according to claim 1, wherein the degradable material comprises a magnesium alloy.

19. The method according to claim 1, wherein time intervals (B) of two adjacent pulses (P) of a pulse bundle PB are shorter than a time interval (C) between a last pulse P of a given pulse bundle (PB) and a first pulse (P) of subsequent pulse bundle (PB).
